(19) United States
(12) Patent Application Publication
(10) Pub. No.: US 2013/0184583 A1
(43) Pub. Date: Jul. 18, 2013

(54) ULTRASONIC DIAGNOSTIC APPARATUS, MEDICAL IMAGE PROCESSING APPARATUS, AND MEDICAL IMAGE PROCESSING METHOD

(71) Applicants: KABUSHIKI KAISHA TOSHIBA, Tokyo (JP); TOSHIBA MEDICAL SYSTEMS CORPORATION, Otawara-shi (JP)

(72) Inventors: Cong YAO, Otawara-shi (JP); Naohisa KAMIYAMA, Utsunomiya-shi (JP); Tetsuya KAWAGISHI, Nasushiobara-shi (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/735,711

(22) Filed: Jan. 7, 2013

(30) Foreign Application Priority Data
Jan. 16, 2012 (JP) 2012-006036

(51) Int. Cl. A61B 8/08; A61B 8/00; A61B 8/14

(52) U.S. Cl. CPC A61B 8/5207; A61B 8/14; A61B 8/4444; A61B 8/5269; A61B 8/463; A61B 8/488; A61B 8/5246
USPC 600/441; 600/443

(57) ABSTRACT

According to one embodiment, a ultrasonic transmission/reception unit transmits/receives ultrasonic waves to/from a subject through the ultrasonic probe and generates an echo signal relating to a scan surface. An image generation unit generates an ultrasonic image relating to the scan surface based on the echo signal. A filter executes filter processing with respect to the ultrasonic image and extracts image constituent elements. A feature information generation unit generates feature information indicative of an amount of change in number of the extracted image constituent elements with respect to a change in characteristics of the filter.

### CROSS-REFERENCE TO RELATED APPLICATIONS

This application is based upon and claims the benefit of priority from Japanese Patent Application No. 2012-006036, filed Jan. 16, 2012, the entire contents of which are incorporated herein by reference.

### FIELD

Embodiments described herein relate generally to an ultrasonic diagnostic apparatus, a medical image processing apparatus, and a medical image processing method.

### BACKGROUND

An ultrasonic diagnostic apparatus is a diagnostic apparatus that displays an image of intravital information; it is inexpensive and free from danger of radiation exposure as compared with other image diagnostic apparatuses such as an X-ray diagnostic apparatus or an X-ray computed tomographic apparatus, and utilized as a useful apparatus for non-invasive observation in real time. The ultrasonic diagnostic apparatus can be widely applied, and it is applied to a diagnosis of a circulatory organ such as the heart, an abdominal part such as the liver or a kidney, a peripheral blood vessel, obstetrics and gynecology, or breast cancer.

Meanwhile, in orthopedics or a diagnosis of rheumatism and the like, generally, bloodstream information of a region of interest is acquired by using the ultrasonic diagnostic apparatus, and a health condition of a subject is ascertained through an amount and a conformation of the bloodstream.

In recent years, an amount of bloodstream in a region of interest is represented by, for example, the number of pixels expressing a bloodstream signal (the number of pixels constituting a bloodstream present region) in an ultrasonic image (for example, a color Doppler image or a power Doppler image) including the bloodstream signal representing that a rate or power of the bloodstream is not lower than a fixed value.

### BRIEF DESCRIPTION OF THE DRAWINGS

FIG. 1 is a view showing a block configuration of an ultrasonic diagnostic apparatus 10 according to an embodiment;

FIG. 2 is a view for explaining the detail of an image generation circuit 24 depicted in FIG. 1;

FIG. 3 is a view for explaining the detail of an image processing unit;

FIG. 4 is a flowchart showing a processing procedure of the image processing unit 30 depicted in FIG. 3;

FIG. 5 is a view showing an example of a three-dimensional ultrasonic Doppler image obtained when a finger of a subject P who is a normal person is determined as a region of interest;

FIG. 6 is a view showing an example of a three-dimensional ultrasonic Doppler image obtained when a finger of a subject P who is a rheumatic is determined as a region of interest; and

FIG. 7 is a view showing an example of a graph produced when image processing is executed on the ultrasonic Doppler images shown in FIG. 5 and FIG. 6.

### DETAILED DESCRIPTION

In general, according to one embodiment, an ultrasonic diagnostic apparatus comprises an ultrasonic probe, an ultrasonic transmission/reception unit, an image data generation unit, a filter, and a feature information generation unit. The ultrasonic transmission/reception unit transmits/receives ultrasonic waves to/from a subject through the ultrasonic probe and generates an echo signal relating to a scan surface. The image generation unit generates an ultrasonic image relating to the scan surface based on the echo signal. The filter executes filter processing with respect to the ultrasonic image and extracts image constituent elements. The feature information generation unit generates feature information indicative of an amount of change in number of the extracted image constituent elements with respect to a change in characteristics of the filter.

The ultrasonic diagnostic apparatus according to this embodiment will now be described hereinafter with reference to the drawings. It is to be noted that, in the following description, like reference numerals denote constituent elements having substantially the same functions and structures, and a duplicate description will be given only when it is necessary.

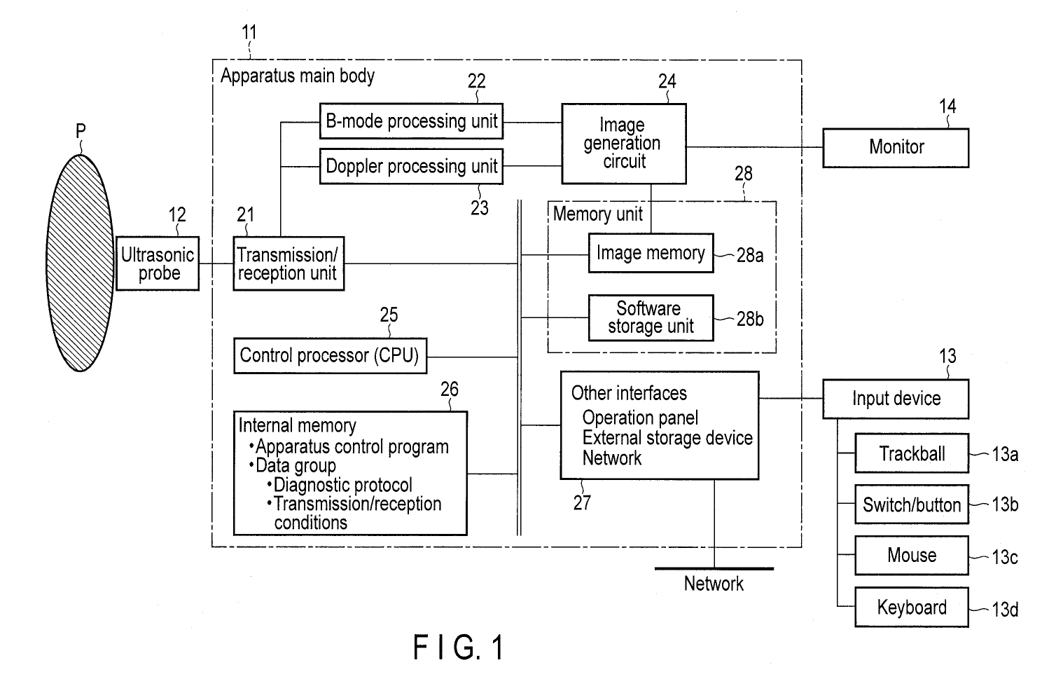

FIG. 1 is a view showing a block configuration of the ultrasonic diagnostic apparatus 10 according to this embodiment. As shown in FIG. 1, the ultrasonic diagnostic apparatus 10 comprises an ultrasonic diagnostic apparatus main body (which will be simply referred to as an apparatus main body hereinafter) 11, an ultrasonic probe 12, an input device 13, and a monitor 14. Further, the apparatus main body (a medical image processing apparatus) 11 comprises a transmission/reception unit 21, a B-mode processing unit 22, a Doppler processing unit 23, an image generation circuit 24, a control processor (CPU) 25, an internal memory 26, an interface unit 27, and a memory unit 28 having an image memory 28a and a software storage unit 28b. It is to be noted that the transmission/reception unit 21 or the like included in the apparatus main body 11 is constituted of hardware such as an integrated circuit in some cases, but it may be a software program modularized in terms of software. A function of each constituent element will now be described hereinafter.

The ultrasonic probe 12 has piezoelectric vibrators each of which generates an ultrasonic wave based on a drive signal from the transmission/reception unit 21 and converts a reflected wave from a subject P into an electrical signal, matching layers provided to the piezoelectric vibrators, a backing material that prevents the ultrasonic wave from being propagated toward the rear side from the piezoelectric vibrators, and others. When the ultrasonic wave is transmitted from the ultrasonic probe 12 to the subject P, the transmitted ultrasonic wave is sequentially reflected on discontinuous surfaces with acoustic impedances of body tissues, turned to an echo signal, and received by the ultrasonic probe 12. An amplitude of this echo signal is dependent on a difference between acoustic impedances on the discontinuous surfaces where the ultrasonic wave is reflected. Further, an echo when an ultrasonic pulse to be transmitted is reflected on moving bloodstream or a surface of a cardiac wall or the like is subjected to frequency shift while being dependent on a speed component of a moving body in an ultrasonic transmitting direction due to the Doppler effect.

The input device 13 is connected to the apparatus main body 11 and has a trackball 13a, various switches/buttons 13b, a mouse 13c, a keyboard 13d, and others configured to fetch various instructions from an operator, conditions, a setting instruction of a region of interest (ROI), various image quality condition setting instructions, and others.

The monitor 14 displays morphological information or bloodstream information in a biological body as an image based on a video signal from the image generation circuit 24.

The transmission/reception unit 21 has a non-illustrated trigger generation circuit, a delay circuit, a pulsar circuit, and others. The pulsar circuit repeatedly generates a rate pulse configured to form an ultrasonic wave to be transmitted at a predetermined rate frequency fr Hz (a cycle; 1/fr second). Further, the delay circuit supplies a delay time required for converging the ultrasonic wave into a beam shape in accordance with each channel and determining transmission directivity to each rate pulse. The trigger generation circuit applies a drive pulse to the ultrasonic probe 12 at a timing based on this rate pulse.

It is to be noted that the transmission/reception unit 21 has a function that enables instantaneously changing a transmission frequency, a transmission drive voltage, or the like in accordance with an instruction from the control processor 25. In regard to changing the transmission drive voltage in particular, this change can be realized by a linear amplification type transmission circuit that can instantaneously change a voltage or a mechanism that electrically switches power supply units.

Furthermore, the transmission/reception unit 21 has an amplification circuit, an analog-to-digital converter, an adder, and others which are not shown. The amplification circuit amplifies the echo signal fetched through the ultrasonic probe 12 in accordance with each channel. The analog-to-digital converter supplies a delay time required for determining reception directivity with respect to the amplified echo signal, and then the adder executes addition processing. Based on this addition, a reflection component from a direction associated with the reception directivity of the echo signal is emphasized, and a comprehensive beam for ultrasonic transmission/reception is formed based on the reception directional characteristic and the transmission directivity.

The B-mode processing unit 22 receives the echo signal from the transmission/reception unit 21, executes logarithm amplification, envelope demodulation processing, or the like, and generates data that represents signal strength by using luminance. This data is transmitted to the image generation circuit 24 and displayed in the monitor 14 as a B-mode image representing the signal strength by using the luminance.

The Doppler processing unit 23 executes frequency analysis of the rate information from the echo signal received from the transmission/reception unit 21, extracts bloodstream, tissue, and contrast medium echo components obtained by the Doppler effect, and acquires bloodstream information such as an average rate, dispersion, power, and the like at multiple points. The obtained bloodstream information is supplied to the image generation circuit 24 and displayed in the monitor 14 as an average rate image, a dispersion image, a power image, and an image which is a combination of these images in colors.

The image generation circuit 24 converts a scan line signal string of ultrasonic scan into a scan line signal string in a general video format as typified by TV and the like and generates an ultrasonic diagnostic image as a display image. The ultrasonic diagnostic image generated by the image generation circuit 24 includes an ultrasonic Doppler image or the like associated with, for example, a scan surface of the subject P generated based on the echo signal concerning the scan surface. It is to be noted that this ultrasonic Doppler image is an image concerning a rate or power of the bloodstream in the subject P and it includes a bloodstream signal indicating that, for example, a rate or power of the bloodstream in the subject P has a fixed value or a higher value. The image generation circuit 24 has a storage memory that stores image data mounted thereon, and an operator can call an image recorded during an examination after a diagnosis, for example. It is to be noted that data that has not been input to the image generation circuit 24 may be referred to as “raw data” in some cases.

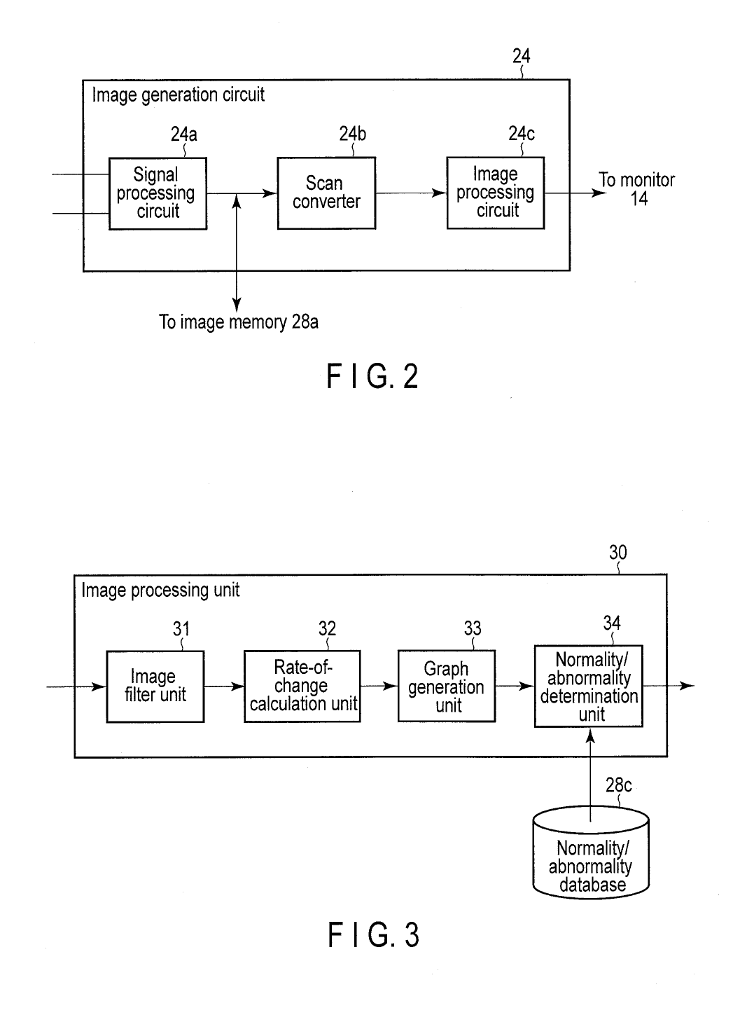

Here, FIG. 2 shows the detail of the image generation circuit 24. As shown in FIG. 2, the image generation circuit 24 includes a signal processing circuit 24a, a scan converter 24b, and an image processing circuit 24c.

First, the signal processing circuit 24a executes filtering for, for example, determining image quality on a scan line level of the ultrasonic scan. An output from the signal processing circuit 24a is supplied to the scan converter 24b and also stored in the image memory 28a in the memory unit 28.

The scan converter 24b converts a scan line signal string of the ultrasonic scan into a scan line signal string in a general video format as typified by TV and the like. An output from the scan converter 24b is transmitted to the image processing circuit 24c.

In the image processing circuit 24c, an output from the scan converter 24b is subjected to image processing for adjustment of luminance or contrast, a space filtering or the like, or combined with character information or scale marks of various setting parameters, or the like, and a resultant signal is output to the monitor 14 as a video signal. In this manner, a tomogram image representing a subject tissue shape is displayed.

The control processor 25 has a function as an information processing device (a computer) and is controlling means that controls an operation of the apparatus main body 11. The control processor 25 reads a control program configured to execute, for example, later-described image processing from the internal memory 26, develops it on the software storage unit 28b in the memory unit 28, and executes arithmetic operations/control or the like concerning various kinds or processing.

The internal memory 26 stores, for example, the above-described control program, diagnostic information (a patient ID, a doctor's remark, and others), a diagnostic protocol, transmission/reception conditions, and any other data groups. Further, the internal memory 26 is used for, for example, storage of images in the image memory 28a as required. Data in the internal memory 26 can be transferred to a peripheral device outside the ultrasonic diagnostic apparatus 10 through an interface unit 27.

The interface unit 27 is an interface concerning the input device 13, a network, and a new external storage device (not shown). Data of an ultrasonic image and the like, an analysis result, and others obtained in the ultrasonic diagnostic apparatus 10 can be transferred to other devices by using the interface unit 27 through the network.

It is to be noted that the image memory 28a is formed of a storage memory that stores various kinds of image data (for example, the ultrasonic Doppler image). This image data can be called by, for example, an operator after a diagnosis, and it can be reproduced like a still image or a moving image that uses pieces of data. Further, the image memory 28a stores an output signal immediately after the transmission/reception unit 21 (which is called a radio-frequency [RF] signal), an image luminance signal obtained after passing through the B-mode processing unit 22 or the Doppler processing unit 23, other raw data, image data acquired through the network, and others as required.

An operation of the ultrasonic diagnostic apparatus 10 according to this embodiment will now be described hereinafter. In the ultrasonic diagnostic apparatus 10 according to this embodiment, image processing for quantitatively extracting, for example, features of a distribution of a bloodstream signal is executed. This image processing is executed by the image processing unit in the ultrasonic diagnostic apparatus 10. It is to be noted that this image processing unit is realized when the control processor 25 executes the control program stored in the internal memory 26.

Here, FIG. 3 shows the detail of the image processing unit. As shown in FIG. 3, the image processing unit 30 includes an image filter unit 31, a rate-of-change calculation unit 32, and a graph generation unit 33.

The image filter unit 31 receives, for example, an ultrasonic Doppler image including a bloodstream signal indicating that a rate or power of the bloodstream is equal to or above a fixed value from the image memory 28a. According to the bloodstream signal included in this ultrasonic Doppler image, a bloodstream present region in a region of interest is shown in the ultrasonic Doppler image. It is to be noted that the ultrasonic Doppler image input by the image filter 31 is a two-dimensional or three-dimensional image.

The image filter unit 31 changes the bloodstream signal included in the input ultrasonic Doppler image by a function of the image processing filter. Alternatively, it changes each pixel displayed as the bloodstream in the ultrasonic Doppler image by the function of the image processing filter. The image filter unit 31 has, for example, a noise reduction filter that executes noise reduction processing (filter processing) with respect to the ultrasonic Doppler image. The ultrasonic Doppler images before and after the noise reduction processing are transferred to the rate-of-change calculation unit 32.

The rate-of-change calculation unit 32 compares the ultrasonic Doppler images before and after the noise reduction processing. As a result, the rate-of-change calculation unit 32 calculates a rate of change in the number of image constituent elements constituting the bloodstream present region included in each ultrasonic Doppler image (i.e., the number of image constituent elements representing the bloodstream signal in the ultrasonic Doppler image) caused due to the noise reduction processing. It is to be noted that the number of image constituent elements constituting the bloodstream present region included in the ultrasonic Doppler image is the number of voxels (three-dimensional pixels) constituting the bloodstream present region when the ultrasonic Doppler image is a three-dimensional image, and it is the number of pixels constituting the bloodstream present region when the ultrasonic Doppler image is a two-dimensional image. Moreover, the rate-of-change calculation unit 32 calculates a rate of change associated with of filter characteristics of the noise reduction filter. The rate of change calculated by the rate-of-change calculation unit 32 is transferred to the graph generation unit 33.

The graph generation unit 33 generates feature information (for example, a graph) indicating dependence of the rate of change calculated by the rate-of-change calculation unit 32 with respect to a change in filter characteristics of the noise reduction filter (an amount of change with respect to a change in characteristics of the filter). The feature information generated by the graph generation unit 33 is displayed in the monitor 14 in the form of an index of a numerical value or a graphic form.

Here, although omitted in FIG. 1, the memory unit 30 has a normality/abnormality database 28c. This normality/abnormality database 28c stores feature information that serves as a reference with respect to the feature information generated by the graph generation unit 33 in advance.

A normality/abnormality determination unit 34 compares the feature information generated by the graph generation unit 33 with the feature information that is stored in the normality/abnormality database 28c and serves as a reference and thereby determines whether the feature information generated by the graph generation unit 33 (i.e., the subject P from which the ultrasonic Doppler image used for generating the feature information is obtained) is normal or abnormal. It is to be noted that the determination result obtained by the normality/abnormality determination unit 34 is displayed in, for example, the monitor 14.

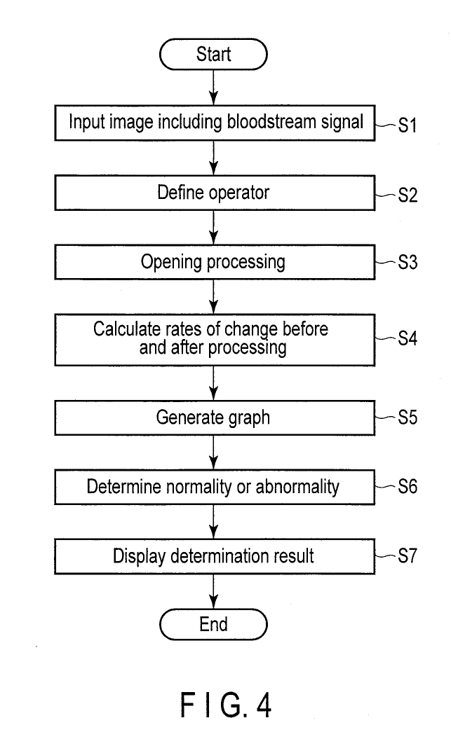

A processing procedure of the image processing unit 30 shown in FIG. 3 will now be described with reference to a flowchart in FIG. 4.

First, the image filter unit 31 included in the image processing unit 30 receives, for example, an ultrasonic Doppler image including the bloodstream signal stored in the image memory 28a (step S1). It is to be noted that the ultrasonic Doppler image received by the image filter unit 31 is a three-dimensional image in the following description.

Then, the image filter unit 31 executes the noise reduction processing with respect to (the bloodstream signal included in) the ultrasonic Doppler image input through the noise reduction filter.

The noise reduction processing executed by the image filter unit 31 will now be specifically explained. In the following description, as the noise reduction processing, opening processing in the morphological operation (which will be simply referred to as opening processing hereinafter) is executed. It is to be noted that the morphological operation is processing used for various kinds of image processing such as feature extraction of an image or noise reduction.

In this case, the image filter unit 31 defines an operator (a structural element representing a range where an operation is executed in the opening processing) required in the opening processing (step S2). At this time, the image filter unit 31 defines, for example, a size and a shape of the operator. Here, it is assumed that, as the size and the shape of the operator, a spherical voxel having a predetermined radius (for example, a radius 1) is defined. The image filter unit 31 uses the defined operator and executes the opening processing with respect to the ultrasonic Doppler image (step S3). In the opening processing, for example, each finer bloodstream structure than the defined operator (a portion of the bloodstream present region represented by the bloodstream signal) is weighted, and the bloodstream structure is removed from the ultrasonic Doppler image. That is, the ultrasonic Doppler image after execution of the opening processing is an image from which a part (i.e., noise) of the bloodstream present region included in the ultrasonic Doppler image before execution of the opening processing has been removed.

Then, the rate-of-change calculation unit 32 compares the ultrasonic Doppler images before and after the opening processing and calculates a rate of change of the number of voxels (a bloodstream count) constituting the bloodstream present region included in the ultrasonic Doppler image (step S4). In this case, the rate of change of the number of voxels constituting the bloodstream present region is represented as “the number of voxels constituting the bloodstream present region included in the ultrasonic Doppler image after the opening processing/the number of voxels constituting the bloodstream present region included in the ultrasonic Doppler image before the opening processing” when the number of voxels constituting the bloodstream present region included in the Ultrasonic Doppler image before the opening processing is normalized as 1. It is to be noted that the thus calculated rate of change is hardly affected by selection of, for example, a position of the region of interest.

It is to be noted that, when the definition of the operator (the size and the shape of the operator) is changed, a degree of removal of the bloodstream present region in the ultrasonic Doppler image varies. In this embodiment, it is assumed that such a definition of an operator (filter characteristics) is appropriately changed and the rate of change is calculated in accordance with each definition of the operator. In other words, the processing of steps S2 to S4 is repeated in accordance with, for example, each definition of the operator.

Then, the graph generation unit 33 generates, for example, a graph (which will be referred to as a subject graph hereinafter) as feature information representing dependence of the rate of change calculated by the rate-of-change calculation unit 32 with respect to a change in definition of the operator (the filter characteristics) (step S5). In the subject graph, the rate of change calculated for each of the definitions of the operator is shown.

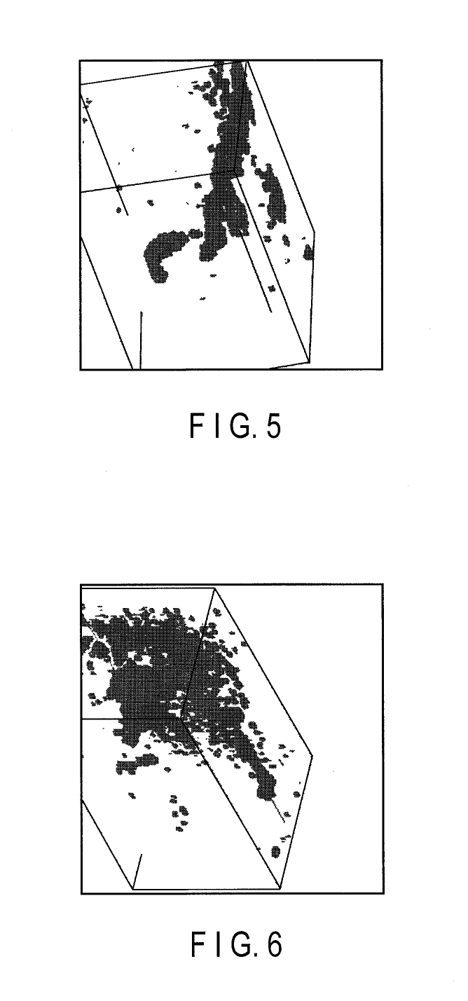

Here, FIG. 5 shows an example of a three-dimensional ultrasonic Doppler image (which will be referred to as an ultrasonic Doppler image of a normal person) obtained when a finger of the subject P who is the normal person is determined as a region of interest. Further, FIG. 6 shows an example of a three-dimensional ultrasonic Doppler image (which will be referred to as an ultrasonic Doppler image of a rheumatic hereinafter) obtained when a finger of the subject P who is a rheumatic is determined as a region of interest.

As shown in FIG. 5 and FIG. 6, a bloodstream present region (a bloodstream signal) included in the ultrasonic Doppler image of the rheumatic extensively spreads as compared with the bloodstream present region included in the ultrasonic Doppler image of the normal person due to, for example, inflammation of a rheumatic region (namely, there are many fine structures). That is, the bloodstream of the rheumatic (the bloodstream present region included in the ultrasonic Doppler image of the rheumatic) is characterized in that it is in disorder as compared with that of the normal person.

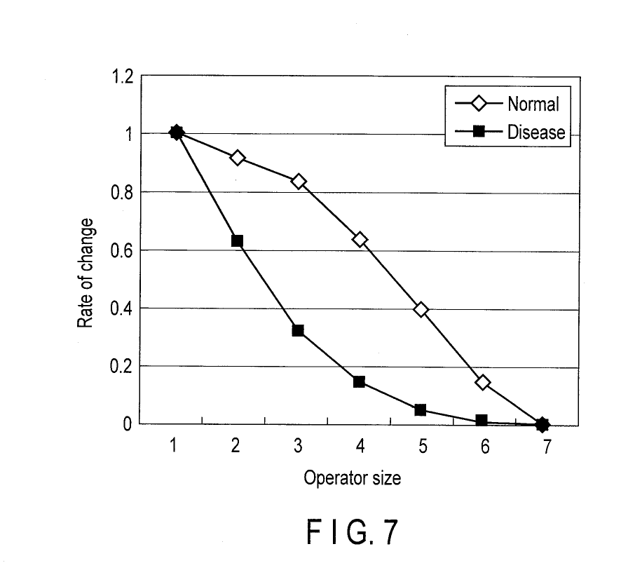

FIG. 7 shows an example of a graph generated when the processing of steps S1 to S5 (the image processing) is executed with respect to the ultrasonic Doppler images shown in FIG. 5 and FIG. 6. It is to be noted that FIG. 7 shows each rate of change when the size of the operator (here, a radius) is changed from 1 to 7.

As described above, since the bloodstream present region included in the ultrasonic Doppler image of the rheumatic is in disorder as compared with the bloodstream present region included in the ultrasonic Doppler image of the normal person (i.e., conformations of the bloodstream are different), when the size of the operator (the radius) is changed from 1 to 7, different curves are drawn depending on the normal person and the rheumatic as shown in FIG. 7. Specifically, since many fine portions of the bloodstream present region (bloodstream structures) are present in the ultrasonic Doppler image of the rheumatic, even if the size of the operator is relatively small, these structures are removed by the opening processing. Therefore, in the graph generated from the ultrasonic Doppler image of the rheumatic, the rate of change calculated as described intensively varies from a lower level of the operator size and the rate of change more rapidly approximates zero as compared with the graph generated from the ultrasonic Doppler image of the normal person.

As described above, in this embodiment, when the image processing is executed, the features of the distribution of the bloodstream present region (the bloodstream signal) included in the ultrasonic Doppler image can be quantitatively extracted as shown in FIG. 7.

Again referring to FIG. 4, the normality/abnormality determination unit 34 acquires (a graph representing) feature information that is stored in the normality/abnormality database 28c and serves as a reference. It is to be noted that the graph (which will be referred to as a reference graph hereinafter) that is stored in the normality/abnormality database 28c and serves as a reference includes a graph (which will be referred to as a normality graph or an abnormality graph hereinafter) generated by, for example, executing the processing of steps S1 to S5 with respect to the three-dimensional ultrasonic

Doppler image of the normal or abnormal subject.

The normality/abnormality determination unit 34 compares the subject graph with the acquired reference graph and thereby determines whether a curve (i.e., the subject P) drawn in the graph generated by the graph generation unit 33 is normal or abnormal (step S6). For example, in a case where the reference graph is a normal graph, it can be determined that the subject P is normal if the subject graph is similar to the normal graph, or it can be determined that the subject P is abnormal if the subject graph is largely different from the normal graph. On the other hand, in a case where the reference graph is, for example, an abnormal graph, if the subject graph is similar to the abnormal graph, it can be determined that the subject P is abnormal.

As a specific example, when a correlation coefficient of the normal graph and the subject graph is calculated and a correlation coefficient of the abnormal graph and the subject graph is likewise calculated, it is possible to determined normality or abnormality in accordance with the graph having the calculated high correlation coefficient.

It is to be noted that, in the normality or abnormality determination processing in step S6, an absolute quantitative index (for example, a rate of change of the normalized number of voxels) may be used, or a relative quantitative index (for example, an area sandwiched between the subject graph and the reference graph) may be used.

A result of the determination made by the normality/abnormality determination unit 34 is displayed in, for example, the monitor 14 (step S7).

Although a result of the determination made by the normality/abnormality determination unit 4 is displayed in step S7 in the above description, Only the rate of change calculated in step S4 or the graph (the subject graph) generated in step S5 as a parameter for determining whether the subject P is normal or abnormal may be displayed in the monitor 14, or the subject graph and the reference graph may be displayed so that they can be compared with each other.

It is to be noted that, in this embodiment, the ultrasonic Doppler image input by the image filter unit 31 is a three-dimensional image in the description, but the ultrasonic Doppler image may be a two-dimensional image. In this case, a predetermined range (constituent elements) may be defined as a size and a shape of an operator so that a rate of change of the number of pixels constituting a bloodstream present region included in each of the ultrasonic Doppler images before and after the opening processing can be calculated.

Furthermore, in this embodiment, the opening processing in the morphological operation is executed as the noise reduction processing using the noise reduction filter in the above explanation, but processing other than the opening processing may be executed as the noise reduction processing, and it is possible to execute processing by using an image filter that can extract features of a shape (a distribution) of the bloodstream signal such as a low-pass filter based on Fourier transformation (in the case of the low-pass filter, the operator is a cutoff frequency) or a smoothing filter for images. Moreover, it is also possible to adopt a threshold filter that hides a flow rate value or a power value that is not greater than a threshold value (in the case of the threshold filter, the operator is a cutoff threshold value), an erosion filter using the morphological operation, a median filter that substitutes an intermediate value in a kernel domain for a pixel value at the center in the kernel domain (in the case of the median filter, the operator is in a kernel range), and others. It is to be noted that, according to the erosion filter, there is carried out processing for leaving only pixels that overlap when an image A is arranged to overlap an image A′ obtained by moving the image A for a length corresponding to several pixels in a direction of the constituent elements. In the case of this erosion filter, the operator is a moving length of the image A′ corresponding to pixels.

Additionally, in this embodiment, although the feature information representing an amount of change in the number of image constituent elements with respect to a change in characteristics of the filter is generated by varying the definition of the operator in the opening processing that is executed with respect to the ultrasonic Doppler image in the above description, other parameters may be varied as a change in characteristics of the filter. Specifically, for example, it is possible to change transmission power or a frequency when the ultrasonic wave is transmitted from the ultrasonic probe 12 or a temperature on a scan surface of the subject P. In this case, there is generated feature information indicative of an amount of change in the number of image constituent elements (for example, the number of voxels) constituting a bloodstream present region included in the ultrasonic Doppler image with respect to such a change.

It is to be noted that, in regard to a frequency when the ultrasonic wave is transmitted from the ultrasonic probe 12, for example, a center frequency can be changed in the range of 2 MHz to 6 MHz.

Further, a temperature on a scan surface of the subject P can be changed by, for example, providing a heater to the ultrasonic probe 12 or utilizing heat generation when the ultrasonic probe 12 operates. It is to be noted that a temperature on the scan surface of the subject P can be detected by, for example, disposing a temperature sensor to the ultrasonic probe 12 in advance.

Furthermore, in the ultrasonic diagnostic apparatus 10, there is a technology for carrying out one set of ultrasonic transmission/reception, which is repeatedly performed while reversing a phase polarity on the same scan line, or one set of ultrasonic transmission/reception, which is repeatedly performed while changing an amount of amplitude modulation, on the same scan line more than once, combining a plurality of sets of reflection wave data received as a result of the ultrasonic transmission/reception, and generating an image by using the combined sets of reflection wave data (which will be referred to as a pulse subtraction repeat transmission/reception technology hereinafter). According to this technology, a band corresponding to a frequency band of the transmitted ultrasonic wave is suppressed from the reflection wave data obtained by combining one set of reflection wave data, and a plurality of sets of extracted non-linear component data are combined. Based on this combination of the sets, non-linear component data having an excellent signal-to-noise ratio (an SN ratio)can be extracted.

In the case of the ultrasonic diagnostic apparatus 10 to which this pulse subtraction repeat transmission/reception technology is applied, it is possible to generate feature information indicative of an amount of change in the number of image constituent elements (the number of voxels) constituting a bloodstream present region included in the ultrasonic Doppler image with respect to a change in number of sets of ultrasonic transmission/reception performed based on such a pulse subtraction repeat transmission/reception technology.

Moreover, in this embodiment, although the image constituent elements constituting the bloodstream present region included in the ultrasonic Doppler image can be extracted by filtering the ultrasonic Doppler image in the above description, this embodiment can be applied to ultrasonic images other than the ultrasonic Doppler image. Specifically, image constituent elements (for example, voxels) constituting a region of microstructures included in an ultrasonic image like a B-mode image may be extracted by executing, for example, contrast false alarm rate (CFAR) processing on the ultrasonic image. In this case, feature information indicative of an amount of change in the number of image constituent elements (the number of voxels) with respect to a change in, for example, kernel of the CFAR filter or in transmission power or frequency of the ultrasonic wave is generated. Likewise, image constituent elements (for example, voxels) constituting a region of a predetermined luminance range included in an ultrasonic image like a B-mode image may be extracted by filtering the ultrasonic image. In this case, for example, feature information indicative of an amount of change in the number of image constituent elements (the number of voxels) with respect to a change in luminance range is generated.

That is, this embodiment can be applied to a situation where the number of image constituent elements constituting a specific region included in an ultrasonic image varies with respect to a comprehensive change in characteristics including, for example, transmission power or a frequency of the ultrasonic wave.

As described above, in this embodiment, the ultrasonic wave is transmitted or received to or from the subject through the ultrasonic probe, an echo signal concerning a scan surface is generated, data of an ultrasonic Doppler image concerning a rate or power of bloodstream of the subject associated with the scan surface is generated based on the echo signal, the noise reduction processing is executed with respect to the ultrasonic Doppler image, and feature information indicative of dependence of a rate of change in the number of image constituent elements (the number of voxels or the number of pixels) constituting a bloodstream present region included in the ultrasonic Doppler image caused by the noise reduction processing with respect to a change in filter characteristics of the noise reduction filter is generated, whereby features of a distribution of a bloodstream signal included in the ultrasonic Doppler image can be quantitatively extracted. That is, the feature information generated in this embodiment represents features of a bloodstream conformation (a distribution), the dependence on the selection of a region of interest is low, and hence robustness is higher than that of the number of image constituent elements simply constituting the bloodstream present region.

Additionally, in this embodiment, when the generated feature information is compared with feature information that is stored in the database and serves as a reference, whether the bloodstream conformation of the subject P is normal or abnormal can be determined.

Further, in this embodiment, since the generated feature information is displayed by using an index of a numerical value or a graphic form, an examiner can evaluate the bloodstream conformation of the subject P based on the index.

It is to be noted that, in this embodiment, features of a distribution of the bloodstream signal are quantitatively extracted from an ultrasonic Doppler image obtained by the ultrasonic diagnostic apparatus 10 in the above description, but any other ultrasonic image such as a B-mode image can be adopted as a target. Further, an image obtained by any other diagnostic device such as an X-ray device, a CT device, or an MRI device may be used as a target. That is, this embodiment enables quantitatively extracting features of a distribution of a signal in an image even though this is an image other than an ultrasonic Doppler image obtained by the ultrasonic diagnostic apparatus 10. Furthermore, in general, Doppler scan can detect not only the bloodstream but also “movement of a moving tissue” like a cardiac wall, for example. That is, since not only the bloodstream but also moving objects in general in the subject can be detected in the ultrasonic Doppler image, it is also possible to quantitatively extract features of a distribution of a moving object (i.e., a signal other than the bloodstream signal) that moves in the subject from the ultrasonic Doppler image.

Moreover, in this embodiment, whether the subject P is normal or abnormal is determined by extracting features of a distribution of a bloodstream signal (a bloodstream present region) included in a ultrasonic Doppler image in the above description, but a vascular structure or the like can be determined in accordance with features of the distribution of the bloodstream signal. Specifically, in a case where a degree of noise reduction of the noise reduction filter is increased, if a change in number of pixels in an image is large, it can be determined that “many fine blood vessels are included or fine bloodstream distributions disperse due to inflammation of a tissue”. On the contrary, if a change in number of pixels in the image is small, it can be determined that “many thick blood vessels are included”.

Additionally, in this embodiment, although the reference graph stored in the normality/abnormality database 28c includes a graph which is generated when the processing of steps S1 to S5 is executed with respect to an ultrasonic Doppler image of a normal or abnormal subject in the above description, the reference graph that can serve as a reference for the subject graph can suffice. Specifically, the normality/abnormality database 28c may store, for example, a graph that is generated by executing the same processing on an ultrasonic Doppler image obtained from symmetrical regions of interest (for example, a right knee and a left knee, or a right kidney and a left kidney) with respect to a region of interest from which an ultrasonic Doppler image of the subject P can be acquired. As a result, for example, when features of distributions of bloodstream signals in the corresponding left and right regions of interest in the subject P are compared, normality or abnormality of the subject P can be determined. Furthermore, reference graphs associated with various kinds of disease conditions may be prepared in the normality/abnormality database 28c in advance, and the reference graphs may be compared with the subject graph, whereby a disease condition of the subject P can be identified. Besides, for example, when subject graphs of the same subject are previously generated before and after an surgical operation and these graphs are compared, a degree of prognostic improvement can be determined.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an ultrasonic probe;
an ultrasonic transmission/reception unit which transmits or receives ultrasonic waves to or from a subject through the ultrasonic probe and generates an echo signal relating to a scan surface;
an image generation unit which generates an ultrasonic image relating to the scan surface based on the echo signal;
a filter which executes filter processing with respect to the ultrasonic image and extracts image constituent elements; and
feature information generation unit which generates feature information indicative of an amount of change in number of the extracted image constituent elements with respect to a change in characteristics of the filter.

2. The apparatus according to claim 1,
wherein the filter executes noise reduction processing with respect to the ultrasonic image as the filter processing.

3. The apparatus according to claim 1, further comprising:
a database which previously stores feature information that serves as a reference with respect to the generated feature information; and
a determination unit which compares the generated feature information with the feature information that is stored in the data base and serves as the reference, and determines whether the generated feature information is normal or abnormal based on a result of the comparison.

4. The apparatus according to claim 1, further comprising a display processing unit which displays the generated feature information in the form of an index of a numerical value or a graphic form.

5. The apparatus according to claim 1,
wherein the image generation unit generates an ultrasonic Doppler image indicative of a rate or power of bloodstream of the subject, and
the filter executes the filter processing with respect to the ultrasonic Doppler image and extracts image constituent elements constituting a bloodstream present region included in the ultrasonic Doppler image.

6. The apparatus according to claim 1,
wherein the filter executes contrast false alarm rate (CFAR) processing with respect to the ultrasonic image and extracts image constituent elements constituting a region of microstructures included in the ultrasonic image.

7. The apparatus of claim 1,
wherein the filter executes the filter processing with respect to the ultrasonic image and extracts image constituent elements constituting a region of a predetermined luminance range included in the ultrasonic image.

8. A medical image processing apparatus comprising:
an image storage unit which stores an ultrasonic image;
a filter which executes filter processing with respect to the ultrasonic image and extracts image constituent elements; and
a feature information generation unit which generates feature information indicative of an amount of change in number of the extracted image constituent elements with respect to a change in characteristics of the filter.

9. The apparatus according to claim 8,
wherein the filter executes noise reduction processing with respect to the ultrasonic image as the filter processing.

10. The apparatus according to claim 8, further comprising:
a database which previously stores feature information that serves as a reference with respect to the generated feature information; and
a determination unit which compares the generated feature information with the feature information that is stored in the data base and serves as the reference, and determines whether the generated feature information is normal or abnormal based on a result of the comparison.

11. The apparatus according to claim 8, further comprising a display processing unit which displays the generated feature information in the form of an index of a numerical value or a graphic form.

12. The apparatus according to claim 8,
wherein the ultrasonic image comprises an ultrasonic Doppler image indicative of a rate or power of bloodstream of the subject, and
the filter executes the filter processing with respect to the ultrasonic Doppler image and extracts image constituent elements constituting a bloodstream present region included in the ultrasonic Doppler image.

13. The apparatus according to claim 8,
wherein the filter executes contrast false alarm rate (CFAR) processing with respect to the ultrasonic image and extracts image constituent elements constituting a region of microstructures included in the ultrasonic image.

14. The apparatus according to claim 8,
wherein the filter executes the filter processing with respect to the ultrasonic image and extracts image constituent elements constituting a region of a predetermined luminance range included in the ultrasonic image.

15. A medical image processing method which is executed by a medical image processing apparatus comprising an image storage unit which stores an ultrasonic image, the method comprising:
executing filter processing with respect to the ultrasonic image and extracting image constituent elements; and
generating feature information indicative of an amount of change in number of the extracted image constituent elements with respect to a change in characteristics in the filter processing.

16. The method according to claim 15,
wherein the executing comprises executing noise reduction processing with respect to the ultrasonic image as the filter processing.

17. The method according to claim 15,
wherein the medical image processing apparatus comprises a database which previously stores feature information that serves as a reference with respect to the generated feature information, and
the method further comprises comparing the generated feature information with the feature information that is stored in the data base and serves as the reference, and determining whether the generated feature information is normal or abnormal based on a result of the comparison.

18. The method according to claim 15, further comprising displaying the generated feature information in the form of an index of a numerical value or a graphic form.

19. The method according to claim 15,
wherein the ultrasonic image comprises an ultrasonic Doppler image indicative of a rate or power of bloodstream of the subject, and
the executing comprises executing the filter processing with respect to the ultrasonic Doppler image and extracting image constituent elements constituting a bloodstream present region included in the ultrasonic Doppler image.

20. The method according to claim 15,
wherein the executing comprises executing contrast false alarm rate (CFAR) processing with respect to the ultrasonic image and extracting image constituent elements constituting a region of microstructures included in the ultrasonic image.

21. The method according to claim 15,
wherein the executing comprises executing the filter processing with respect to the ultrasonic image and extracting image constituent elements constituting a region of a predetermined luminance range included in the ultrasonic image.
